(19) United States
(12) Patent Application Publication
(10) Pub. No.: US 2014/0024626 A1
(43) Pub. Date: Jan. 23, 2014

(54) USE OF CANTHAXANTHIN AND/OR 25-OH D3 FOR IMPROVED HATCHABILITY IN POULTRY

(71) Applicant: DSM IP ASSETS B.V., Heerlen (NL)

(72) Inventors: Jose-Maria HERNANDEZ, Magden (CH); Gilbert WEBER, Magden (CH)

(73) Assignee: DSM IP ASSETS B.V., Heerlen (NL)

(21) Appl. No.: 14/034,384

(22) Filed: Sep. 23, 2013

Related U.S. Application Data
(62) Division of application No. 13/128,275, filed on May 9, 2011.

(30) Foreign Application Priority Data
Nov. 19, 2008 (EP) 08020153.6

(51) Int. Cl. A61K 31/122; A61K 31/593

(52) U.S. Cl. CPC A61K 31/122; A61K 31/593
USPC 514/167; 514/691

(57) ABSTRACT

The present invention relates to the use canthaxanthin and/or 25-hydroxy vitamin D3 (25-OH D3) for improving breeder hatchability and fertility and for lowering embryo mortality in poultry. More particularly, the invention relates to the use of Canthaxanthin and/or 25-hydroxy canthaxanthin in the manufacture of a food or veterinary composition for improving hatchability in poultry.

### CROSS-REFERENCE

This application is a divisional of commonly owned copending U.S. application Ser. No. 13/128,275, filed May 9, 2011 (now abandoned), which is the national phase application under 35 USC §371 of PCT/EP2009/064963, filed Nov. 11, 2009, which designated the US and claims priority to European Application No. 08020153.6, filed Nov. 19, 2008, the entire contents of which are hereby incorporated by reference.

### FIELD OF THE INVENTION

The present invention relates to the use of canthaxanthin and/or at least one vitamin D metabolite, preferably 25-hydroxy vitamin D3 (25-OH D3), for improving breeder hatchability. More particularly the invention relates to the use of canthaxanthin and/or 25-hydroxy vitamin D3 in the manufacture of a feed or veterinary composition for improving hatchability in poultry.

### BACKGROUND

To maximize the fertility of the egg and the hatchability of the embryo during the breeding phase, optimal nutritional status of breeders is essential for the effective transfer of nutrients to the embryo.

In accordance with the present invention it has been found that problems in chicken breeding can be eliminated or substantially ameliorated by administering to the animals an effective amount of Canthaxanthin or 25-OH-D3, optionally a combination of both nutrients.

Applicant now surprisingly found that relatively high concentrations of essential nutrients in the embryo such as canthaxanthin or 25-OH-D3 supplied via maternal supplementation are related to improved hatchability, fertility and lower embryo mortality during the first phase of embryo development.

Tritsch et al. (US 2003/0170324) disclose a feed premix composition of at least 25-OH D3 in an amount between 5% and 50% (wt/wt) dissolved in oil and an antioxidant, an agent encapsulating droplets of 25-OH D3 and oil, and a nutritional additive (e.g., Vitamin D3). The premix may be added to poultry, swine, canine, or feline food. This composition stabilizes 25-OH D3 against oxidation.

Simoes-Nunes et al. (US 2005/0064018) discloses adding a combination of 25-OH Vitamin D3 and Vitamin D3 to animal feed. In particular, about 10 μg/kg to about 100 μg/kg of 25-OH Vitamin D3 and about 200 IU/kg to about 4,000 IU/kg of Vitamin D3 are added to swine feed. This addition improves the pig's bone strength.

Stark et al. (U.S. Pat. No. 5,695,794) disclose adding a combination of 25-OH Vitamin D3 and Vitamin D3 to poultry feed to ameliorate the effects of tibial dyschondroplasia.

Borenstein et al U.S. Pat. No. 5,043,170 discloses the combination of Vitamin D3 and either 1-alpha-hydroxycholecalciferol or 1alpha, 25-dihydroxycholecalciferol to improve egg strength and leg strength in laying hens and older hens.

Fleshner-Barak (WO 03/007916) discloses administration of bisphosphonate compound and natural vitamin D derivative such as 1,25-dihydroxyvitamin D3 or 24,25-dihydroxyvitamin D3, or 25-OH vitamin D3.

Daifotis et al. (WO 03/086415) disclose inhibiting bone resorption by a combination of at least one bisphosphonate compound and from about 100 IU to about 60,000 IU of a no activated metabolite of vitamin D2 and/or vitamin D3.

The aforementioned documents did not teach or suggest that the use of canthaxanthin and 25-OH D3 or a combination thereof would be surprisingly beneficial to improve hatchability.

### DETAILED DESCRIPTION OF THE INVENTION

As used throughout the specification and claims, the following definitions apply:

“Vitamin D metabolite” means any metabolite of Vitamin D as for example 25-hydroxy vitamin D3, 1,25-dihydroxy vitamin D3 or 24,25-dihydroxy vitamin D3.

“25-OH D3” refers specifically to 25-hydroxy vitamin D3.

“Poultry” is meant to include turkeys, ducks and chickens (including but not limited to broiler chicks, layers, breeders).

Canthaxanthin and 25-OH D3 may be obtained from any source, and a composition thereof may be prepared using convenient technology.

In a first aspect, one or more feed compositions suitable for poultry use are provided to administer canthaxanthin or 25-OH D3 and combinations thereof as nutrients to improve hatchability, fertility and lower embryo mortality during the first phase of embryo development.

In a second aspect, a poultry feed is provided which comprises from about 10 μg/kg to about 100 μg/kg of 25-OH D3 and/or from about 2 to 100 ppm canthaxanthin, preferably 2 to 10 ppm.

In another aspect, a method of administering canthaxanthin and/or 25-OH D3 to poultry breeders is provided to improve hatchability, fertility and lower embryo mortality during the first phase of embryo development.

The method for improving hatchability in poultry comprises administering to the animal in need of such treatment an amount of about 2 ppm to 100 ppm of canthaxanthin, preferably 2 to 10 ppm, and/or about 10 μg/kg to about 100 μg/kg of 25-OH D3.

In another aspect, a premix composition for poultry feed comprising 25-hydroxy vitamin D3 and canthaxanthin is provided.

Canthaxanthin and 25-hydroxy vitamin D3 are suitably administered together with the food. The term food as used herein comprises both solid and liquid food as well as drinking fluids such as drinking water. Particularly, inventive ingredients can be added as a formulated powder to a premix containing other minerals, vitamins, amino acids and trace elements which is added to regular animal food and thorough mixing to achieve even distribution therein.

In the manufacture of poultry feed in accordance with the invention, from about 2 ppm to 100 ppm, preferably 2-10 ppm of canthaxanthin and, if required, from about 10 μ/kg to about 100 μg/kg of 25-hydroxy vitamin D3 are added to regular poultry food. Alternatively, a food premix may be prepared on the basis of regular food components by adding these active ingredients to such food components in higher concentration.

According to the present invention the canthaxanthin compound is available under the Trademark ROVIMIX®Hy-D® 1.25% and canthaxanthin under the Trademark CAROPHYLL®Red.

According to the present invention it is further advantageous if the composition also contains one or more of the following ingredients: Vitamin A, Vitamin E, Biotin, copper (e.g. as CuSO4), zinc (e.g. as ZnSO4), cobalt (e.g. as CoSO4), selenium (e.g. as Na2SeO3), iodine (e.g. as KI), manganese (e.g. as MnSO4) and/or calcium (e.g. as CaSO4).

The following non-limiting Examples are presented to better illustrate the invention.

### EXAMPLE 1

### Effect of Carophyll Red (Canthaxanthin) on the Productive and Reproductive Development of Broiler Chickens

### Material & Methods

In this study 360 females and 36 male broiler chickens were used, all 45 weeks of age, and of Cobb 500 lineage. The birds were housed together in their respective treatment groups according to body weight and the uniformity of the batch.

Pre-experiment phase—37th to 45th week:

In the pre-experiment phase, the birds underwent the recommended handling and feeding practices in the breeders' guide. To assess the fertility of the birds in each box, an incubation session was carried out for one week, and using embryo diagnostics on the eggs that did not hatch, the percentage of fertility in each box was determined. The level of fertility was taken into consideration when distributing the treatments to each of the boxes, so that all treatments had the same level of fertility at the beginning of the study.

Period of Experimentation—46th to 66th weeks:

At the start of this period, all the birds were weighed and this was repeated every 28 days during the period that the birds received the treatment (Table 1). During the experiment, information was collected on the daily production of eggs. Sample of the birds were weighed on a weekly basis.

### Feeding

The diet given to the birds was a standard feed for broiler chickens with the addition of the products that were tested. The feed met all nutritional requirements in relation to the developmental stage of the birds and the recommendations in the breeders' guide. The feed was entirely vegetable-based, using corn and Soya bran (Appendices 1, 2 and 3).

TABLE 1
Treatments used in the experiment on broiler chickens (lineage - Cobb
500) for a period of six months.

| Treatments | Carophyll Red (ppm) |
|---|---|
| 1 | 0 |
| 2 | 60 |

### Experimental Design

The experimental design was entirely random, with two treatments and six groups of 30 female and 3 male chickens.

### Methodology

The laying rate was calculated weekly. To assess the weight of the eggs, specific gravity, average weight of eggs, yolk weight, albumen weight and the coloration of the yolk, all the eggs that were not considered suitable for incubation, collected on any given day, were used. Specific gravity was determined through the emersion of the eggs in saline solutions with densities of 1065; 1070; 1075; 1080; 1085; 1090 and 1095. The weighing of the eggs, yolks and albumen were carried out using a precision weighing scale (0.001 g). The coloration of the yolks was determined using the color fan from DSM Nutritional Products®.

To evaluate hatching, hatchability, fertility and embryonic mortality the eggs were collected daily. They were then classified and marked with the number of the corresponding box. Those that were not considered suitable for incubation were stored for a maximum period of seven days in an air-conditioned room with temperature and humidity control. Incubation was carried out in a multi-stage incubator and on day 18, the eggs were transferred to a brooder. On day 21 the chicks were taken out of the brooder, vaccinated and classified. The eggs that did not hatch then underwent embryo diagnostics to evaluate fertility and the phase of embryonic mortality.

### Statistical Analysis

After the data was obtained, a variation analysis was carried and standard deviation was calculated. These statistical procedures were carried out with the help of the statistical program SAS.

### Results

TABLE 2
Laying rate during the periods 46th-55th, 56th-66th weeks and total period
(21 weeks)

| | Laying rate (%) | | |
|---|---|---|---|
| Treatments | 46th to 55th weeks | 56th to 66th week | Total period |
| Control | 58.19 ± 3.87 | 48.75 ± 3.19 | 53.25 ± 2.68 |
| Carophyll Red | 59.66 ± 2.97 | 51.35 ± 3.66 | 55.31 ± 2.22 |
| Arithmetic Mean | 58.93 | 50.05 | 54.28 |
| C.V. (%) | 5.86 | 6.87 | 4.54 |
| P | 0.4781 | 0.2207 | 0.1793 |

TABLE 3
Hatchability in the periods between the 46th-56th and 56th-66th weeks
and the total period (21 weeks)

| | Hatchability (%) | | |
|---|---|---|---|
| | | | average |
| Treatments | 46th to 55th week | 56th to 66th week | of the 21 weeks |
| Control | 92.48 ± 0.98 b | 93.42 ± 0.51 b | 92.97 ± 0.54 b |
| Carophyll Red | 94.33 ± 0.76 a | 95.96 ± 0.53 a | 95.18 ± 0.56 a |
| Arithmetic Mean | 93.41 | 94.69 | 94.08 |
| C.V. (%) | 0.94 | 0.55 | 0.59 |
| P | 0.0047 | 0.0001 | 0.0001 |

(a > b Duncan's test)

TABLE 4
Hatching, hatchability, fertility, and embryonic mortality during the observed
period (21 weeks)

| | Hatching | Hatchability | Fertility | Embryonic Mortality |
|---|---|---|---|---|
| Treatments | (%) | | | |
| Control | 83.03 ± 0.89 b | 92.97 ± 0.54 b | 90.98 ± 0.81 b | 5.46 ± 0.75 a |
| Carophyll Red | 86.03 ± 0.42 a | 95.18 ± 0.56 a | 92.11 ± 0.48 a | 3.72 ± 0.86 b |
| Arithmetic | 84.53 | 94.08 | 91.54 | 4.59 |
| Mean | | | | |
| C.V. (%) | 0.83 | 0.59 | 0.75 | 16.77 |
| P | 0.0001 | 0.0001 | 0.0171 | 0.0029 |

(a > b Duncan's test)

TABLE 5
Effect of the treatments on the average embryonic mortality rate over the
observed period

| | Embryonic Mortality (%) | | | |
|---|---|---|---|---|
| Treatments | M1 | M2 | M3 | M4 |
| Control | 1.80 ± 0.45a | 0.89 ± 0.70 | 0.69 ± 0.28 | 2.07 ± 0.23 a |
| Carophyll | 1.04 ± 0.41b | 0.66 ± 0.33 | 0.58 ± 0.33 | 1.44 ± 0.58 b |
| Red | | | | |
| Arithmetic | 1.42 | 0.77 | 0.64 | 1.76 |
| Mean | | | | |
| C.V. (%) | 28.02 | 40.41 | 54.07 | 21.86 |
| P | 0.0083 | 0.2225 | 0.5980 | 0.0171 |

(a > b Duncan's test)
M1—Embryonic Mortality in the first 48 hours of incubation
M2—Embryonic Mortality occurring between day 3 and day 7 of incubation
M3—Embryonic Mortality occurring between day 8 and day 14 of incubation
M4—Embryonic Mortality occurring between day 15 and day 21 of incubation

TABLE 6
Embryonic Mortality between the 19th and 21st weeks and for all 21
weeks of the study

| | Embryonic Mortality (%) | | | |
|---|---|---|---|---|
| Treatments | Week 19 | Week 20 | Week 21 | 21 weeks |
| Control | 5.32 ± 3.24 | 4.57 ± 1.57 | 5.18 ± 2.72 | 5.46 ± 0.75 a |
| Carophyll | 3.33 ± 4.12 | 3.51 ± 2.56 | 3.12 ± 2.12 | 3.72 ± 0.86 b |
| Red | | | | |
| Arithmetic | 4.33 | 4.04 | 4.15 | 4.59 |
| Mean | | | | |
| C.V. (%) | 57.93 | 52.75 | 54.88 | 16.77 |
| P | 0.0127 | 0.4081 | 0.1490 | 0.0029 |

(a > b Duncan's test)

TABLE 7
Number of eggs, incubatible eggs and chicks per bird housed during the
observed period (21 weeks)

| | | Incubatible | |
|---|---|---|---|
| Treatments | Production of eggs/bird | eggs/bird | Chicks/bird |
| Control | 78.27 ± 3.95 | 71.71 ± 4.46 | 5952 ± 3.18 b |
| Carophyll Red | 81.30 ± 3.40 | 75.25 ± 3.46 | 64.73 ± 2.82 a |
| Arithmetic | 79.79 | 73.48 | 62.13 |
| Mean | | | |
| C.V. (%) | 4.55 | 5.36 | 4.76 |
| P | 0.1793 | 0.1503 | 0.0122 |

TABLE 8
Calculated nutritional levels in the feed used during the experiment

| | | | |
|---|---|---|---|
| Average Metabolic Energy | 2850.00 | Average Metabolic | 2850.00 |
| (Kcal) | | Energy (Kcal) | |
| Gross Protein (%) | 15.96 | Canthaxanthin (UI) | 1662.50 |
| Arginine Total (%) | 0.94 | Vitamin E (mg) | 53.59 |
| Lysine Total (%) | 0.80 | Vitamin K3 (mg) | 2.50 |
| Methionine Total (%) | 0.35 | Vitamin B1 (mg) | 5.77 |
| Methionine + Cystine | 0.55 | Vitamin B2 | 10.82 |
| Total (%) | | (mg) | |
| Threonine Total (%) | 0.59 | Vitamin B6 (mg) | 10.85 |
| Triptophan Total (%) | 0.17 | Vitamin B12 (mcg) | 19.00 |
| Isoleucine Total (%) | 0.63 | Biotin (mg) | 0.30 |
| Leucine Total (%) | 1.46 | Folic Acid (mg) | 1.74 |
| Valine Total (%) | 0.72 | Nicotinic Acid (mg) | 65.36 |
| Histidine Total (%) | 0.42 | Pantothenic Acid (mg) | 25.22 |
| Chlorine Total (%) | 0.77 | Copper (mg) | 18.43 |
| Calcium (%) | 3.30 | Iron (mg) | 122.94 |
| Available Phosphorus (%) | 0.40 | Iodine (mg) | 0.80 |
| Sodium (%) | 0.19 | Manganese (mg) | 83.10 |
| Chlorine (%) | 0.28 | Selenium (mg) | 0.69 |
| Potassium (%) | 0.60 | Zinc (mg) | 93.83 |
| Vitamin A (UI) | 10450.00 | | |

TABLE 9
Composition of feed used during the experiment

| Ingredients | Percentage |
|---|---|
| Corn | 68.52 |
| Soya Bran 46% | 21.57 |
| Limestone 38% Ca | 7.21 |
| Bicalcium Phosphate | 1.64 |
| Salt | 0.4 |
| Wheat Bran | 0.112 |
| DL-Methionine 99% | 0.045 |
| Premix | 0.50 |

TABLE 10
Composition of the premix added to the diet of the chickens

| Nutrient | Quantity per kilo of product | Unit |
|---|---|---|
| Folic Acid | 237.5 | mg |
| Nicotinic Acid | 8500 | mg |
| Pantothenic Acid | 3800 | mg |
| Biotin | 38 | mg |
| Copper | 12400 | mg |
| Choline | 72000 | mg |
| Sulphur | 10222 | mg |
| Iron | 12000 | mg |
| Iodine | 160 | mg |
| Manganese | 14000 | mg |
| Methionine | 118800 | mg |
| Oxytetracycline | 8000 | mg |
| Selenium | 108 | mg |
| Vitamin A | 2090000 | UI/Kg |
| Vitamin B1 | 475 | mg |
| Vitamin B12 | 3800 | mg |
| Vitamin B2 | 1900 | mg |
| Vitamin B6 | 950 | mg |
| Canthaxanthin | 332500 | UI/Kg |
| Vitamin E | 7600 | mg |
| Vitamin K3 | 950 | mg |
| Zinc | 14000 | mg |

### Example 2

### Study if the Supplementation of Canthaxanthin and 25-OH D3 in Broiler Breeder Hens

### Material & Methods:

The test has been executed according to Example 1, with the addition of ROVIMIX®Hy-D®. The treatments in example 2 were as follows:
T1—Control diet
T2—Control diet+60 ppm of Carophyll Red
T3—Control diet+69 ppm of ROVIMIX®Hy-D®
T4—Control diet+60 ppm of Carophyll Red+69 ppb of ROVIMIX®Hy-D® and with 6 replication/treatment of 40 broiler breeder and 4 cockerels per replication.

Egg production, fertility and hatchability are recorded weekly. The results are shown in Table 11. The data shows a good response for ROVIMIX®Hy-D® and canthaxanthin.

TABLE 11

| TRT | Average hatchability after 10 weeks |
|---|---|
| Control | 85.26 |
| ROVIMIX ® Hy-D ® | 86.85 |
| CAROPHYLL RED | 87.22 |
| CAROPHYLL RED + | 88.07 |
| ROVIMIX ® Hy-D ® | |

1. A method for improving poultry hatchability and fertility and for lowering poultry embryo mortality comprising administering to poultry in need of such treatment an amount of about 2 ppm to 100 ppm of canthaxanthin effective to improve poultry hatchability and fertility and lower poultry embryo mortality, and observing the result of administration.

2. The method of claim 1, which comprises administering the canthaxanthin in an amount of about 2 to 10 ppm.

3. The method of claim 1, which further comprises administering the canthaxanthin together with a vitamin D metabolite.

4. The method of claim 3, wherein the vitamin D metabolite is 25-hydroxy vitamin D3.
